(19) United States
(12) Patent Application Publication
(10) Pub. No.: US 2018/0028632 A1
(43) Pub. Date: Feb. 1, 2018

(54) METHOD OF TREATING TUMORS WITH NK AND NKT CELLS EXPRESSING ANTI-SSEA4 CHIMERIC ANTIGEN RECEPTORS

(71) Applicant: Lan Bo Chen, Lexington, MA (US)

(72) Inventor: Lan Bo Chen, Lexington, MA (US)

(21) Appl. No.: 15/664,129

(22) Filed: Jul. 31, 2017

Related U.S. Application Data
(60) Provisional application No. 62/368,645, filed on Jul. 29, 2016.

(51) Int. Cl. A61K 39/00; C07K 14/705; A61K 39/395; C07K 14/725; A61K 47/68; C07K 16/18

(52) U.S. Cl. CPC A61K 39/0011; A61K 47/6813; C07K 16/18; A61K 39/39558; C07K 14/7051; C07K 14/70578; C07K 14/70521; C07K 2317/622; C07K 2317/24; C07K 2317/64; C07K 2317/31; C07K 2319/74; A61K 2039/5156

(57) ABSTRACT

Two methods for treating a tumor are disclosed. The first method includes (i) obtaining NK cells or NKT cells from a subject, (ii) transducing the NK cells or NKT cells in vitro with an expression vector encoding a chimeric antigen receptor that contains an scFv specifically recognizing stage-specific embryonic antigen 4, (iii) expanding the transduced NK cells or NKT cells in vitro, and (iv) infusing the expanded transduced NK cells or NKT cells into the subject. The second method includes, in place of step (ii) above, transducing the NK cells or NKT cells in vitro with an expression vector encoding a chimeric antigen receptor that is specific for a tumor antigen other than stage-specific embryonic antigen 4, and further requires a step of administering an antibody against stage-specific embryonic antigen 4.

### CROSS-REFERENCE TO RELATED APPLICATIONS

The present application claims priority to Provisional Application No. 62/368,645, filed on Jul. 29, 2016. The content of this prior application is hereby incorporated by reference in its entirety.

### BACKGROUND

Targeted cancer immunotherapy, as compared to chemotherapy, holds the promise of better efficacy, both short-term and long-term, with fewer side effects.

Recently, chimeric antigen receptors (“CARs”) have been developed to program T cells to attack cells bearing the tumor antigen. A CAR contains (i) an extracellular domain that binds to the tumor antigen and (ii) one or more intracellular domains that provide both primary and co-stimulatory signals to the T cells. T cells can be engineered in vitro to express CAR having an extracellular domain of choice.

The CAR approach has proven to be effective, yet not without serious side effects. In an example, infusion of large numbers of T cells expressing CAR causes graft-versus-host disease, in which the T cells attack non-malignant tissues.

In a different approach, NK cells or NKT cells have been used as anti-cancer agents. These two types of cells, part of the innate immune system, can recognize tumor cells as they arise.

Stimulation of NK cells can lead to a strong anti-tumor response. For example, treatment of acute myeloid leukemia patients with alloreactive NK cells substantially increased their survival without any associated graft-versus-host disease. See Ruggeri et al. 2002, Science 295:2097-2100. On the other hand, NK cells can be inhibited by the tumor microenvironment, thus limiting their effectiveness. See Pegram et al. 2011, Immunol. Cell Biol. 89:216-224.

Turning to NKT cells, activating them with natural ligands, e.g., α-galactosyl-ceramide, presented by dendritic cells, leads to cytokine release which in turn can activate anti-tumor T cells and NK cells. See Van Kaer et al. 2015, Frontiers Immunol. 6:1-11. Yet, overstimulation of NKT cells can result in uncontrolled cytokine release and sepsis. See Id.

There is a need to develop CAR-based tumor therapies that are more effective than those currently in use.

### SUMMARY

To address this need, methods for treating a tumor are provided.

One method includes (i) obtaining NK cells or NKT cells from a subject having a tumor; (ii) transducing the NK cells or NKT cells in vitro with an expression vector encoding a chimeric antigen receptor (CAR) containing an endodomain from CD3ζ or from FcεRIγ fused to a scFv that specifically recognizes stage-specific embryonic antigen 4 (SSEA4), (iii) expanding the transduced NK cells or NKT cells in vitro, and (iv) infusing the expanded transduced NK cells or NKT cells into the subject.

Also disclosed is a second method for treating a tumor. This method includes (i) obtaining NK cells or NKT cells from a subject having a tumor, (ii) transducing the NK cells or NKT cells in vitro with an expression vector encoding a CAR containing an endodomain from CD3ζ or from FcεRIγ fused to a scFv that recognizes an antigen on the tumor, (iii) expanding the transduced NK cells or NKT cells in vitro, (iv) infusing the expanded transduced NK cells or NKT cells into the subject, and (v) administering an antibody that specifically binds to SSEA4, whereby an anti-tumor immune response is raised.

The details of one or more embodiments of the invention are set forth in the description below. Other features, objects, and advantages of the invention will be apparent from the description and from the claims.

Importantly, all documents cited herein are hereby incorporated by reference in their entirety.

### DETAILED DESCRIPTION

As mentioned above, the first method of the invention includes a step in which an expression vector encoding a CAR is transduced in vitro into NK cells or NKT cells, i.e., step (ii).

The encoded CAR contains a scFv that specifically binds to SSEA4. Examples of a scFv that specifically binds to SSEA4 are described in US Patent Application Publication 2016/0102151.

In addition to the scFv, the CAR also contains an endodomain from CD3ζ or FcεRIγ. The endodomain contains one or more immunoreceptor tyrosine-based activating motifs (“ITAM”).

Of note, the CAR further includes a hinge/spacer region and a transmembrane region between the scFv and the endodomain.

Exemplary sequences that can be used as a hinge/spacer region are derived from the hinge region of, e.g., IgG1, IgG4, and IgD. Alternatively, it can be derived from CD8. See, e.g., Dai et al. 2016, J. Natl. Cancer Inst. 108:1-14 (“Dai et al.”) and Shirasu et al., 2012, Anticancer Res. 32:2377-2384 (“Shirasu et al.”).

Exemplary transmembrane regions that can be included in the CAR are derived from CD3, CD4, CD8, or CD28. See Dai et al. and Shirasu et al.

Optionally, the CAR contains a second endodomain in addition to the endodomain from CD3ζ or FcεRIγ. The second endodomain, e,g., from CD28, CD137, CD4, OX40, ICOS, Ly49D, Ly49H, KIR2DL4, KIR2DS1, KIR2DS2, KIR2DS3, KIR2DS4, KIR2DS5, KIR3DS1, NKG2C, NKG2E, NKG2D, NKp30, NKp44, NKp46, NKp80, DNAM-1, or PILR. These endodomains contain one or more ITAM.

Moreover, the CAR can contain a third endodomain, which also can be from CD28, CD137, CD4, OX40, ICOS, Ly49D, Ly49H, KIR2DL4, KIR2DS1, KIR2DS2, KIR2DS3, KIR2DS4, KIR2DS5, KIR3DS1, NKG2C, NKG2E, NKG2D, NKp30, NKp44, NKp46, NKp80, DNAM-1, or PILR. The third endodomain is different from the second endodomain.

In a specific embodiment, the CAR contains an anti-SSEA4 scFv fused to a spacer/hinge from CD8 that is fused to a transmembrane domain also from CD8 fused to the N-terminus of the endodomain from CD28, which in turn is fused to the N-terminus of the endodomain from CD137, which in turn is fused to the N-terminus of the endodomain from CD3ζ.

The expression vector mentioned above includes a promoter operably linked to a nucleic acid encoding the CAR. The promoter is active either in NK cells or in NKT cells.

Exemplary CAR expression vectors based on lentiviral vectors or gamma retroviral vectors are set forth in Dai et al.; Jin et al. 2016, EMBO Mol. Med. 8:702-711; Liechtenstein et al. 2013, Cancers 5:815-837; and Schonfeld et al. 2015, Mol. Therapy 23:330-338 (“Schonfeld et al.”).

Such expression vectors are used for integrating the promoter/CAR-encoding nucleic acid into NK cell or NKT cell genomic DNA to produce stable expression of the CAR.

In an alternative embodiment, the expression vector contains sequences that facilitate transposon-mediated genomic integration of the promoter/CAR-encoding nucleic acid into NK cells or NKT cells. Examples of these expression vectors include, but are not limited to, the so-called “PiggyBac” and “Sleeping Beauty” expression vectors. See Nakazawa et al. 2011, Mol. Ther. 19:2133-2143 and Sourindra et al. 2013, J. Immunotherapy 36:112-123.

The first method of the invention includes obtaining NK cells or NKT cells from a subject suffering from a tumor. Procedures for isolating these cells from the blood are known in the art. See, e.g., Kaiser et al. 2015, Cancer Gene Therapy 22:72-78 (“Kaiser et al.”).

Alternatively, established NK cell lines can be used in the method. See, e.g., Schonfeld et al.

The NK cells or NKT cells are transduced in vitro with a CAR containing a scFv that specifically recognizes SSEA4, i.e., any of the CAR described above. Transduction of NK cells or NKT cells can be performed by electroporation, lipofection, lentiviral infection, or gamma retrovirus infection.

The transduced cells are expanded in vitro, using methods known in the art. See Kaiser et al.

Finally, the expanded NK cells or NKT cells are infused either in one batch or in two or more batches into the subject having a tumor.

In one embodiment, the above-described method of the invention includes a preconditioning step that is performed prior to the just-mentioned infusion step. The preconditioning step is accomplished by treating the subject with a drug that induces lymphodepletion. Examples of such a drug include cyclophosphamide and fludarabine. Additional drug examples can be found in Dai et al. and Han et al. 2013, J. Hematol. Oncol. 6:47-53.

This method for treating a tumor can also include administering an antibody or antibody fragment that specifically binds to SSEA4. Examples of an antibody that specifically binds to SSEA4 include a chimeric anti-SSEA4 antibody and a fully humanized anti-SSEA4 monoclonal antibody. An antibody fragment that specifically binds to SSEA4 can be, but is not limited to, an anti-SSEA4 Fab and an anti-SSEA4 scFv. See US Patent Application Publication 2016/0102151 for more examples of anti-SSEA4 antibodies and anti-SSEA4 antibody fragments for use in this first method of the invention.

Note that the anti-SSEA4 antibody or antibody fragment can be linked to a cytokine, a cytotoxic agent, a modified immunoglobulin Fc domain, anti-CD3, or anti-CD16 in order to improve their ability to stimulate an immune response to tumor cells.

A cytokine can be fused to the anti-SSEA4 antibody or antibody fragment as part of a fusion protein. See Kiefer et al. 2016, Immunol. Revs. 270:178-192. In another example, the cytokine is linked to the anti-SSEA4 antibody or antibody fragment via cross-links between lysine residues. Exemplary suitable cytokines include G-CSF, GM-CSF, IFNγ, IFNα, IL-1β, IL-2, IL-4, IL-6, IL-7, IL-9, IL-12, IL-13, IL-15, IL-17, IL-21, IL-23, and TNF.

Exemplary cytotoxic agents are diphtheria toxin, pseudomonas exotoxin A (“PE38”), doxorubicin, methotrexate, an auristatin, a maytansine, a calicheamicin, a duocarmycin, a pyrrolobenzodiazepine dimer, and 7-ethyl-10-hydroxy-camptothecin. Suitable cytotoxic agents are described in Peters et al. 2015, Biosci. Rep. 35:1-20 (“Peters et al”); Bouchard et al. 2014, Bioorg. Med. Chem. Lett. 24:5357-5363; Panowski et al. 2014, mAbs 6:34-45; and Mazor et al. 2016, Immunol. Revs. 270:152-164.

The cytotoxic agent can be linked to the anti-SSEA4 antibody or antibody fragment via a linker. In an embodiment, the linker is cleavable such that, upon internalization of the antibody or antibody fragment by a tumor cell, the cytotoxic agent is cleaved from the antibody or antibody fragment. Examples of a cleavable linker include, but are not limited to, acid-labile small organic molecules (e.g., hydrazone), protease cleavable peptides (e.g., valine-citrulline dipeptide), and disulfide bonds. In another embodiment, the linker is not cleavable. In this case, the cytotoxic agent is released upon degradation of the anti-SSEA4 antibody or antibody fragment linked to it. Additional examples of linkers are described in Peters et al.

If the cytotoxic agent is a protein, it can be linked to the anti-SSEA4 antibody or antibody fragment via a peptide bond, e.g., as part of a fusion protein. In a particular example, PE38 can be fused to the C-terminus of a V_{L} chain of an anti-SSEA4 monoclonal antibody.

In a particular embodiment, the first method for treating a tumor is carried out by administering an anti-SSEA4 antibody fragment linked to a modified immunoglobulin Fc domain together with the CAR-expressing NK cells or NKT cells. For example, the Fc domain can be modified such that it specifically targets the FcγRIIa receptor, the FcγRIIIa receptor, or the FcRn receptor, as compared to an unmodified Fc domain. Targeting the FcγRIIa or FcγRIIIa receptor leads to an increased cytotoxic immune response. On the other hand, targeting the FcRn receptor increases the half-life of the anti-SSEA4 antibody fragment. Modifications to the Fc domain that increase its affinity for the FcγRIIa receptor, the FcγRIIIa receptor, or the FcRn receptor are described in Moore et al. 2010, mAbs 2:181-189 and Lobner et al. 2016, Immunol. Revs. 270:113-131.

In another embodiment, the anti-SSEA4 antibody fragment is linked to an anti-CD3 molecule. The anti-CD3 molecule activates T cells localized to tumor cells via the anti-SSEA4 antibody fragment. An exemplary anti-CD3 molecule is an antibody fragment. The anti-CD3 molecule can specifically bind to CD3ε. Further, a scFv that specifically binds to SSEA4 can be fused to another scFv that specifically binds to CD3.

In still another embodiment, the anti-SSEA4 antibody fragment is linked to an anti-CD16 molecule. The anti-CD16 molecule activates NK cells localized to tumor cells via the anti-SSEA4 antibody fragment. Like the anti-CD3 molecule described in the preceding paragraph, the anti-CD16 molecule can be an antibody fragment that binds specifically to CD16. Exemplary constructs are an anti-SSEA4/anti-CD16 chimeric antibody and a scFv that specifically binds to SSEA4 fused to another scFv that specifically binds to CD16.

The first method above for treating a tumor is effective for treating e.g., a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

Turning to the second method of this invention, it can also be used for treating the tumors mentioned in the preceding paragraph.

The second method requires the steps of obtaining NK cells or NKT cells from a subject having a tumor, transducing the NK cells or NKT cells in vitro with an expression vector that encodes a CAR, expanding the transduced NK cells or NKT cells in vitro, infusing the expanded transduced NK cells or NKT cells into the subject, and administering an antibody that specifically binds to SSEA4.

This method employs an expression vector that encodes a CAR having a target different than SSEA4, the CAR target used in the first method. The CAR utilized in the second method specifically binds to the following targets: α-folate receptor, CD19, CD20, CAIX, CD22, CD30, CD33, CD44v7/8, CEA, EGP-2, EGP-40, erb-B2, erb-B3, erb-B4, FBP, fetal acetylcholine receptor, GD2, GD3, Her2/neu, IL-13R-α2, KDR, kappa light chain, LeY, L1, MAGE-A1, mesothelin, MUC1, NKG2D ligand, h5T4, PSCA, PSMA, TAG-72, or VEGF-R2. The specific CAR is selected depending on the presence of the target in the tumor to be treated. For example, a CAR that specifically binds to CD19 can be used for treating a B-cell tumor.

The second method uses the same procedures for obtaining, transducing, expanding, and infusing the NK cells or NKT cells into the subject as the first method. The second method, like one embodiment of the first method, includes administering an antibody that specifically binds to SSEA4. The antibody can be linked to a cytokine, a cytotoxic agent, a modified immunoglobulin Fc domain, anti-CD3, and anti-CD16 as set forth above.

Without further elaboration, it is believed that one skilled in the art can, based on the description above, utilize the present invention to its fullest extent.

The following references, some cited supra, can be used to better understand the background of the application:
Abate-Daga et al., Mol. Ther. Oncolytics 3:1-7.
Bouchard et al. 2014, Bioorg. Med. Chem. Lett. 24:5357-5363
Becker et al. 2010, J. Immunol. 184:6822-6832
Curran et al. 2012, J. Gene Med. 14:405-415
Dai et al. 2016, J. Natl. Cancer Inst. 108:1-14
Guest et al., 2005, J. Immunother. 28:203-211
Han et al. 2013, J. Hematol. Oncol. 6:47-53
Heczey et al. 2014, Blood 124:2824-2833
James et al. 2008, J. Immunol. 180:7028-7038.
Kaiser et al. 2015, Cancer Gene Therapy 22:72-78.
Kiefer et al. 2016, Immunol. Revs. 270:178-192
Lawson 2012, Immunology 137:20-27
Lobner et al. 2016, Immunol. Revs. 270:113-131
Mazor et al. 2016, Immunol. Revs. 270:152-164
Moore et al. 2010, mAbs 2:181-189
Moritz et al. 1995 Gene Therapy 2:539-546
Nakazawa et al. 2011, Mol. Ther. 19:2133-2143
Panowski et al. 2014, mAbs 6:34-45
Pegram et al. 2011, Immunol. Cell Biol. 89:216-224
Peters et al. 2015, Biosci. Rep. 35:1-20
Rajagopalan et al. 2005, J. Exp. Med. 201:1025-1029
Rezvani et al. 2015, Front. Immunol. 17 November
Rodgers et al. 2016, Proc. Natl. Acad. Sci. January 12:E459-E468
Ruggeri et al. 2002, Science 295:2097-2100
Schonfeld et al. 2015, Mol. Therapy 23:330-338
Shirasu et al. 2012, Anticancer Res. 32:2377-2384
Sourindra et al., 2013, J. Immunotherapy 36:112-123
Van Kaer et al. 2015, Frontiers Immunol. 6:1-11

The contents of the above references are hereby incorporated by reference in their entirety.

### Other Embodiments

All of the features disclosed in this specification may be combined in any combination. Each feature disclosed in this specification may be replaced by an alternative feature serving the same, equivalent, or similar purpose. Unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method for treating a subject having a tumor, the method comprising:
obtaining NK cells or NKT cells from the subject;
transducing the NK cells or NKT cells in vitro with an expression vector encoding a chimeric antigen receptor (CAR) containing an endodomain from CD3ζ or from FcεRIγ fused to a scFv that specifically recognizes stage-specific embryonic antigen 4 (SSEA4), whereby the transduced NK cells or NKT cells express the CAR;
expanding the transduced NK cells or NKT cells in vitro; and
infusing the expanded transduced NK cells or NKT cells into the subject, whereby an anti-tumor immune response is raised.

2. The method of claim 1, wherein the CAR further contains a second endodomain from CD28, CD137, CD4, OX40, ICOS, Ly49D, Ly49H, KIR2DL4, KIR2DS1, KIR2DS2, KIR2DS3, KIR2DS4, KIR2DS5, KIR3DS1, NKG2C, NKG2E, NKG2D, NKp30, NKp44, NKp46, NKp80, DNAM-1, or PILR.

3. The method of claim 2, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

4. The method of claim 3, further comprising administering an antibody or antibody fragment that specifically binds to SSEA4.

5. The method of claim 4, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

6. The method of claim 5, wherein the antibody or antibody fragment is linked to a cytokine, a cytotoxic agent, a modified immunoglobulin Fc domain, anti-CD3, or anti-CD16.

7. The method of claim 6, wherein the antibody or antibody fragment is linked to a cytokine selected from the group consisting of G-CSF, GM-CSF, IFNγ, IFNα, IL-1β, IL-2, IL-4, IL-6, IL-7, IL-9, IL-12, IL-13, IL-15, IL-17, IL-21, IL-23, and TNF.

8. The method of claim 6, wherein the antibody or antibody fragment is linked to a cytotoxic agent selected from the group consisting of Diphtheria toxin, Pseudomonas exotoxin A, doxorubicin, methotrexate, an auristatin, a maytansine, a calicheamicin, a duocarmycin, a pyrrolobenzodiazepine dimer, and 7-ethyl-10-hydroxy-camptothecin.

9. The method of claim 6, wherein the antibody or antibody fragment is linked to an immunoglobulin Fc domain modified to target the FcγRIIa receptor, the FcγRIIIa receptor, or the FcRn receptor.

10. The method of claim 6, wherein the antibody fragment is linked to anti-CD3 or anti-CD16.

11. A method for treating a subject having a tumor, the method comprising:
obtaining NK cells or NKT cells from the subject;
transducing the NK cells or NKT cells in vitro with an expression vector encoding a chimeric antigen receptor (CAR) containing an endodomain from CD3ζ or from FcεRIγ fused to a scFv that specifically recognizes an antigen on the tumor, whereby the transduced NK cells or NKT cells express the CAR;
expanding the transduced NK cells or NKT cells in vitro;
infusing the expanded transduced NK cells or NKT cells into the subject, and administering an antibody that specifically binds to SSEA4, whereby an anti-tumor immune response is raised.

12. The method of claim 11, wherein the scFv specifically binds to α-folate receptor, CD19, CD20, CAIX, CD22, CD30, CD33, CD44v7/8, CEA, EGP-2, EGP-40, erb-B2, erb-B3, erb-B4, FBP, fetal acetylcholine receptor, GD2, GD3, Her2/neu, IL-13R-α2, KDR, kappa light chain, LeY, L1, MAGE-A1, mesothelin, MUC1, NKG2D ligand, h5T4, PSCA, PSMA, TAG-72, or VEGF-R2.

13. The method of claim 12, wherein the CAR further contains a second endodomain from CD28, CD137, CD4, OX40, ICOS, Ly49D, Ly49H, KIR2DL4, KIR2DS1, KIR2DS2, KIR2DS3, KIR2DS4, KIR2DS5, KIR3DS1, NKG2C, NKG2E, NKG2D, NKp30, NKp44, NKp46, NKp80, DNAM-1, or PILR.

14. The method of claim 13, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

15. The method of claim 13, wherein the antibody that specifically binds to SSEA4 is linked to a cytokine, a cytotoxic agent, a modified immunoglobulin Fc domain, anti-CD3, or anti-CD16.

16. The method of claim 15, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

17. The method of claim 16, wherein the antibody is linked to a cytokine selected from the group consisting of G-CSF, GM-CSF, IFNγ, IFNα, IL-1β, IL-2, IL-4, IL-6, IL-7, IL-9, IL-12, IL-13, IL-15, IL-17, IL-21, IL-23, and TNF.

18. The method of claim 16, wherein the antibody is linked to a cytotoxic agent selected from the group consisting of Diphtheria toxin, Pseudomonas exotoxin A, doxorubicin, methotrexate, an auristatin, a maytansine, a calicheamicin, a duocarmycin, a pyrrolobenzodiazepine dimer, and 7-ethyl-10-hydroxy-camptothecin.

19. The method of claim 16, wherein the antibody is linked to an immunoglobulin Fc domain modified to target the FcγRIIa receptor, the FcγRIIIa receptor, or the FcRn receptor.

20. The method of claim 16, wherein the antibody is linked to anti-CD3 or anti-CD16.
